(19) United States
(12) Patent Application Publication
(10) Pub. No.: US 2004/0258773 A1
(43) Pub. Date: Dec. 23, 2004

(54) Composition and methods for treating subjects with hyperglycemia

(76) Inventors: Xiaozhuo Chen, Athens, OH (US); Fang Liu, Athens, OH (US); Yungsheng Li, Athens, OH (US); Jing Li, Athens, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/487,904

(22) PCT Filed: Sep. 3, 2002
(86) PCT No.: PCT/US02/27996

(51) Int. Cl. A61K 35/78; A61K 31/7024

(52) U.S. Cl. 424/725; 514/23; 424/774

(57) ABSTRACT

A partially purified plant extract for treating subjects who are hyperglycemic is provided. The partially-purified plant extract comprises a plurality of water soluble compounds having a molecular weight greater than 800 and less than 1500 grams/mole. One of the compounds in the partially-purified plant extract is the hydrolysable gallotannin known as penta-O-galloyl-D-glucose. The partially-purified plant extract is substantially free of triterpenoids, including corosolic acid and high molecular weight ellagitannins, i.e., ellagitannins having a molecular weight of greater than 1500 grams/mole. In a highly preferred embodiment, the partially purified plant extract is derived from the tropical plant Lagerstroemia Speciosa. L, which is also known as banaba. The present invention also relates to a method of using the present partially-purified extract to treat subjects who are hyperglycemic.

This application claims priority to U.S. provisional application 60/316,624 filed on Aug. 31, 2002, which is incorporated herein in its entirety.

### FIELD OF THE INVENTION

The present invention relates to compositions and methods for treating subjects who are hyperglycemic, particularly subjects with Type II diabetes, more particularly diabetic subjects who are overweight.

### BACKGROUND OF THE INVENTION

Diabetes mellitus, commonly called diabetes, refers to a disease process derived from multiple causative factors and characterized by elevated levels of plasma glucose, a condition referred to as hyperglycemia. According to the American Diabetes Association, diabetes mellitus is estimated to affect approximately 6% of the world population. Uncontrolled hyperglycemia is associated with increased and premature mortality due to an increased risk for microvascular and macrovascular diseases, including nephropathy, neuropathy, retinopathy, hypertension, cerebrovascular disease and coronary heart disease. Therefore, control of hyperglycermia is an important approach for the treatment of diabetes.

There are two major forms of diabetes: Type 1 diabetes (formerly referred to as insulin-dependent diabetes or IDDM); and Type 2 diabetes (formerly referred to as noninsulin dependent diabetes or NIDDM). Type I diabetes is the result of an absolute deficiency of insulin, the hormone which regulates glucose utilization. Such absolute insulin deficiency is usually characterized by α-cell destruction within the Islets of Langerhans in the pancreas. Type 2 diabetes is a disease characterized by insulin resistance accompanied by relative, rather than absolute, insulin deficiency. Type 2 diabetes can range from predominant insulin resistance with relative insulin deficiency to predominant insulin deficiency with some insulin resistance. Insulin resistance is the diminished ability of insulin to exert its biological action across a broad range of concentrations. In insulin resistant individuals the body secretes abnormally high amounts of insulin to compensate for this defect. When inadequate amounts of insulin are present to compensate for insulin resistance and adequately control glucose, a state of impaired glucose tolerance develops. In a significant number of individuals, insulin secretion declines further and the plasma glucose level rises, resulting in the clinical state of diabetes.

The majority of Type 2 diabetic patients are treated either with hypoglycemic agents which act by stimulating release of insulin from beta cells, or with agents that enhance the tissue sensitivity of the patients towards insulin, or with insulin. Sulfonylureas are examples of agents that stimulate release of insulin from beta cells. Among the agents applied to enhance tissue sensitivity towards insulin, metformin is a representative example. Even though sulfonylureas are widely used in the treatment of type II diabetes, this therapy is, in most instances, not satisfactory. In a large number of type II diabetic patients sulfonylureas do not suffice to normalize blood sugar levels and the patients are, therefore, at high risk for acquiring diabetic complications. Also, many patients gradually lose the ability to respond to treatment with sulfonylureas and are, thus, gradually forced into insulin treatment. This shift of patients from oral hypoglycemic agents to insulin therapy is usually ascribed to exhaustion of the pancreatic β cells in type II diabetic patients.

Insulin stimulates glucose uptake by skeletal muscle and adipose tissues primarily through translocation of the glucose transporter 4 (GLUT4) from the intracellular storage sites of the cell surface (Saltiel, A. R. & Kahn, C. R. (2001) Nature 414:799-806; Saltiel, A. & Pessin, J. E. (2002) Trends in Cell Biol. 12:65-71; White, M. F. (1998) Mol. Cell. Biochem. 182:3-11). In response to insulin, a fraction of GLUT4 present in intracellular membranes is redistributed to the plasma membrane resulting in an increase of GLUT4 on the cell surface and enhanced glucose uptake by these cells. GLUT4 translocation is primarily mediated through the insulin receptor (IR).

In addition to glucose transport, insulin is intimately involved in adipogenesis, a process which involves proliferation of preadipocytes (pre-fat cells) and differentiation of preadipocytes into adipocytes (fat cells) with accumulation of fat in adipocytes. Studies with the adipocyte cell line 3T3-L1 suggest that the role insulin plays in adipogenesis is primarily mitotic. Before differentiation, 3T3-L1 cells are fibroblast-like preadipocytes that contain more IGF-1 receptors than IR. In vitro, adipogenesis of preadipocytes can be triggered by a commonly used differentiation-inducing cocktail, MDI, which consists of an agent methylisobutylxanthine (MIX) that elevates cAmp; a glucocorticoid, dexamethasone (DEX); and insulin (or IGF-1) that interacts with the IGF-1 receptors on the preadipocytes (Tong, Q., Hotamisligil, G. S. (2001) Rev. in Endoc. & Metabolic Disorders. 2:349-355; Rosen, E. D., et al. (2000) Genes Dev. 14:1293-1307). When treated with MDI, confluent preadipocytes re-enter the cell cycle and undergo approximately two rounds of mitosis (Modan-Moses, D., et al. (1998) Biochem. J 333:825-831; Tong, Q., Hotamisligil, G. S. (2001) Rev. in Endoc. & Metabolic Disorders. 2:349-355; Rosen, E. D., et al. (2000) Genes Dev. 14:1293-1307), a process commonly referred to as clonal expansion. Following clonal expansion, the preadipocytes exit the cell cycle and begin to differentiate into adipocytes by expressing adipocyte genes including C/EBP-α, β, δ, and PPAR-γ.

As a result of its adipogenic effect, insulin has the undesirable effect of promoting obesity in patients with type 2 diabetes. (See, Moller, D. E. (2001) Nature 414:821-827) Unfortunately, other anti-diabetic drugs which are currently being used to stimulate glucose transport in patients with type 2 diabetes also possess adipogenic activity. Thus while current drug therapy may provide reduction in blood sugar, it often promotes obesity. Accordingly, new compositions and methods for treating hyperglycemia are desirable. Compositions that stimulate glucose uptake with generating concomitant adipogenic side effects are especially desirable.

### SUMMARY OF THE INVENTION

The present invention provides a partially-purified plant extract for treating subjects who are hyperglycemic. The partially-purified plant extract comprises a plurality of water soluble compounds having a molecular weight greater than 800 and less than 1500 grams/mole. One of the compounds in the partially purified plant extract is the hydrolysable gallotannin known as penta-O-galloyl-D-glucose. The partially purified plant extract is substantially free of triterpenoids, including corosolic acid and high molecular weight ellagitannins, i.e., ellagitannins having a molecular weight of greater than 1500 grams/mole. In a highly preferred embodiment, the partially purified plant extract is derived from the tropical plant Lagerstroemia Speciosa. L, which is also known as banaba

The present invention also provides a method for reducing blood glucose levels in subjects who are hyperglycemic. The method comprises administering the partially-purified plant extract to the subject. Although it is possible to administer the partially-purified extract to the subject by injection, the preferred method of administration involves oral administration. The method is useful for treating subjects who are hyperglycemic, as well as subjects with diabetes mellitus. The method is especially useful for treating overweight subjects with Type II diabetes.

### BRIEF DESCRIPTION OF THE FIGURES

FIG. 1. The effect of a hot water extract of banaba (BE) on glucose uptake in 3T3-L1 adipocytes. Adipocytes in 12-well plates were incubated for 15 minutes with a hot water extract of banaba (BE), a banaba HP-20 methanol eluent (BME) and a banaba HP-20 column water eluent (BWE), or with insulin as a positive control, or without treatment as a negative control, then assayed for 2-deoxy-D-[³H]glucose uptake. Data are means±SD, n=8. Means with different letters differ, P<0.01.

FIG. 2. The effect of the concentration of a hot water extract of banaba (BE panel A) or insulin (panel B) on glucose uptake in 3T3-L1 adipocytes. Differentiated 3T3-L1 cells in 12-well plates were incubated with different concentrations of BE or insulin for 15 min., and then assayed for 2-deoxy-D-[³H]glucose uptake. Data are means±SEM, n=6.

FIG. 3. Combined effects of insulin and a hot water extract of banaba (BE) on glucose uptake in 3T3-L1 adipocytes. Differentiated 3T3-L1 cells were incubated with insulin in the presence or absence of 0.1 g/l BE (A), or incubated with BE in the presence or absence of 1 μmol/L insulin (B) for 15 min., and then assayed for the glucose uptake activities. Data are means±SEM, n=6.

FIG. 4. The effect of a hot water extract of banaba (BE) on adipocyte differentiation in the absence of insulin in 3T3-L1 cells. Undifferentiated 3T3-L1 preadipocytes were incubated with insulin or BE in the presence of dexamethasone (DEX) and 3-isobutyl-1-methylxanthine (IBMX). After ten days of incubation, the degree of adipocyte differentiation was assayed by the glucose uptake activities of the cells. Data are means±SD, n=6. Means with different letters differ, P<0.01.

FIG. 5. The effect of BE, BWE, and BME on adipocyte differentiation in the presence of insulin (1S) in 3T3-L1 cells. In the presence of dexamethasone (DEX) and 3-isobutyl-1-methylxanthine (IBMX), undifferentiated 3T3-L1 preadipocytes were induced by either insulin, (BE+IS), (BME+IS) or (BWE+IS). After 10 days of incubation, the cells were photographed at magnification ×200. (A) No induction; (B) IS; (C) BE (0.5 g/L) and IS; (D) BME (0.5 g/L) and IS; (E) BWE (0.5 g/L) and IS. Figure shown represents one of four independent experiments. All four experiments showed similar results.

FIG. 6. The effects of introduction time and concentration of banaba hot water extract (BE) on adipocyte differentiation in the presence of insulin (IS) in 3T3-L1 cells. Preadipocyte 3T3-L1 cells were induced with IS in the presence of dexamethasone (DEX) and 3-isobutyl-1-methyl-xanthine (IBMX) at day 0. BE was added to the media either at various times after the initial induction (A) or at various concentrations at the time of the initial induction (B). The degree of differentiation of differently treated 3T3-L1 cells was assayed by the glucose uptake activities of the cells. Data are means±SD, n=6. Means with different letters differ; P<0.01.

FIG. 7. Effect of a methanol (MeOH) extract and a water (H₂O) extract of banaba on glucose uptake in 3T3-L1 adipocytes.

FIG. 8. Effect of a water extract of banaba on adipocyte differentation in 3T3-L1 cells.

FIG. 9. Effect of different concentrations of a water extract of banaba on glucose uptake in 3T3-L1 adipocytes.

FIG. 10. Effects of dexamethasone (DXMS) and 3-isobutyl-1-methylxanthine (IBMX) on adipocyte differentiation in 3T3-L1 cells as measured by glucose uptake.

FIG. 11. Effects of insulin or a water extract of banaba on adipocyte differentiation in 3T3-L1 cells as measured by glucose uptake.

FIG. 12. Inhibition of adipogenesis in 3T3-L1 cells by a water extract of banaba.

FIG. 13. Non-induction of adipocyte differentiation of 3T3-L1 cells at different concentrations of a water extract of banaba as measured by glucose uptake.

FIG. 14. Inhibition of insulin-stimulated adipocyte differentiation in 3T3-L1 cells by a water extract of banaba is time-dependent as measured by glucose uptake.

FIG. 15. Inhibition of insulin-stimulated adipocyte differentiation in 3T3-L1 cells by a water extract of banaba is concentration-dependent.

FIG. 16. Changes of blood glucose level of regular mice fed with 50 mg of glucose with or without a hot water extract of banaba (BE).

FIG. 17. HPLC profile of a hot wafer extract of banaba fractionated by HPLC running method 1. Fractions 1-6 as labeled in the profile were isolated and subsequently assayed for their glucose uptake stimulatory activity.

FIG. 18. Glucose transport-stimulatory activity of fractions 1-6 from HPLC running method 1.

FIG. 19. HPLC profile of a hot water extract of banaba fractionated by HPLC running method 2. Subfractions 1-3 as labeled in this profile were isolated and subsequently assayed for their glucose uptake-stimulatory activities.

FIG. 20. Glucose transport-stimulatory activity of subfractions 1-3 as generated by HPLC running method 2.

FIG. 21. HPLC profile of a hot water extract of banaba fractionated by HPLC running method 3. The F2 and F3 fractions generated by HPLC running method 2 were further separated into single peaks by HPLC running method 3. Several of the individual peaks were isolated (collected) for subsequent glucose-stimulatory activity.

FIG. 22. Glucose transport-stimulatory activity of sub-subfractions SSF12, SSF13, SSF14, SSF 15, SSF16, SSF 18 and SSF 19 as generated by HPLC running method 3.

### DETAILED DESCRIPTION OF THE INVENTION

Definitions

The term “diabetes mellitus” or “diabetes” means a disease or condition that is generally characterized by metabolic defects in production and utilization of glucose which result in the failure to maintain appropriate blood sugar levels in the body. The result of these defects is elevated blood glucose, referred to as “hyperglycemia.” Two major forms of diabetes are Type 1 diabetes and Type 2 diabetes. As described above, Type 1 diabetes is generally the result of an absolute deficiency of insulin, the hormone which regulates glucose utilization. Type 2 diabetes often occurs in the face of normal, or even elevated levels of insulin and can result from the inability of tissues to respond appropriately to insulin. Most Type 2 diabetic patients are insulin resistant and have a relative deficiency of insulin, in that insulin secretion can not compensate for the resistance of peripheral tissues to respond to insulin. In addition, many Type 2 diabetics are obese. Other types of disorders of glucose homeostasis include impaired glucose tolerance, which is a metabolic stage intermediate between normal glucose homeostasis and diabetes. The guidelines for diagnosis for Type 2 diabetes and impaired glucose tolerance have been outlined by the American Diabetes Association (see, e.g., The Expert Committee on the Diagnosis and Classification of Diabetes Mellitus, Diabetes Care, (1999) Vol 2 (Suppl 1): S5-19).

The term “symptom” of diabetes, includes, but is not limited to, polyuria, polydipsia, and polyphagia, hyperinsulinemia, and hyperglycemia as used herein, incorporating their common usage. For example, “polyuria” means the passage of a large volume of urine during a given period; “polydipsia” means chronic, excessive thirst; “polyphagia” means excessive eating, and hyperinsulinemia means elevated blood levels of insulin. Other symptoms of diabetes include, for example, increased susceptibility to certain infections (especially fungal and staphylococcal infections), nausea, and ketoacidosis (enhanced production of ketone bodies in the blood).

Hydrolysable gallotannin” as used herein refers to a galloyl glucose compound that is an ester of glucose with one or more trihydroxybenzene carboxylic acids.

“Triterpenoid” as used herein refers to compounds that are triterpenes or derivatives of triterpenes. One example of a triterpenoid compound that is found in plants is corosolic acid. “Substantially free of triterpenoid compounds” as used herein refers to a partially-purified plant extract that contains less than 1% by weight of one or more triterpenoid compounds. Preferably the partially-purified plant extract of the present invention contains less than 0.5%, more preferably less than 0.1% by weight of one or more triterpenoid compounds.

In one aspect, the present invention provides a partially-purified plant extract, referred to hereinafter as “TE”, for treating subjects with a hyperglycemia, non-insulin-dependent type II diabetes or obesity. In one embodiment, TE is a partially-purified hot water extract of the leaves from the tropical plant Lagerstroemia Speciosa. L, which is also known as banaba. TE comprises a plurality of water soluble, hydrophilic, heat stable, non-protein, non-DNA, non-lipid compounds. One of the compounds in TE is the hydrolysable gallotannin known as penta-O-galloyl-D-glucose. TE further comprises one or more additional compounds having a molecular weight of between 800 and 1500 grams/mole. In accordance with the present invention, it has been determined that a hot water extract of banaba, known hereinafter as “BE”, and TE both stimulate glucose uptake in 3T3-L1 adipocytes without inducing adipocyte differentiation. The glucose-uptake stimulatory activity of BE is both time dependent and concentration dependent. In accordance with the present invention, it has also been determined that blood glucose levels of normal mice fed with glucose are reduced when BE is administered to the mice concurrently with the glucose. In accordance with the present invention, it has also been determined that triterpenoids are not present or are present at significantly reduced amounts in TE.

In one embodiment, TE is isolated from the leaves of the banaba plant according to the following procedure:

1. A hot water extract (HWE) is made by boiling banaba tea in water, preferably for at least 30 minutes.

2. The hot water extract (HWE) is concentrated, preferably by heat evaporation, filtered and centrifuged to remove particulate matter, and, preferably, freeze-dried to form a powder,

3. The powder is then dissolved in water to provide a 10% (w/v) extract solution and separated into fractions by high performance liquid chromatography (HPLC) on a C-18 reversed phase column. Conditions of the HPLC are as follows:

HPLC method for separation of the banaba hot water extract into individual fractions.

HPLC system is Beckman System Gold consisting of a 125 solvent module, a 168 PDA detector and a 508 autosampler. A Beckman Ultrasphere C-18 reversed phase column (10.0 mm×250 mm I.D., 5 μm) was used. The detection wavelengths were set at 210 mi, 300 nm and 370 nm. Eluent A was a mixture of water and 0.1% trifluoroacetic acid, eluent B was a mixture of acetonitrile and 0.1% trifluoroacetic acid. A Foxy Jr. fraction collector from ISCO was used to collect individual fractions in timed windows. Separation was achieved with following linear gradients from A to B in 250 minutes at a flow rate of 2 mL/min

Isocratic A for 25 min, 100:0 to 95:5 in 30 min, isocratic for 25 min, 95:5 to 91.8:8.2 in 2 min, isocratic for 18 min, 91.8:8.2 to 90:10 in 35 min, isocratic for 20 min, 90:10 to 88.5:11.5 in 15 min, 88.5:11.5 to 85:15 in 3 min, isocratic for 27 min, 85:15 to 80:20 in 15 min, isocratic for 15 min, 80:20 to 50:50 in 10 min and then isocratic for 10 min.

In each run, 100 μl of 10% hot water banaba extract is injected using the autosampler. The fraction collection window starts at 40 minutes after injection and ends at 250 minutes. Within the collection window fractions are collected according to the height and width of the peak at wavelength 300 nm (Blue spectra in the HPLC profile but within this window peaks shown at wavelength 300 nm is consistent with those shown at 210 nm (Black spectra). The retention time of each fraction (peak), which has shown positive results in glucose uptake assay, is

| # | 8 | 9 | 12 | 13 | 14 |
|---|---|---|---|---|---|
| t_{R} (min) | 66.0 | 70.0 | 98.2 | 104.0 | 106.5 |
| # | 15 | 16 | 17 | 18 | 19 |
| t_{R} (min) | 110.8 | 138.0 | 141.6 | 147.8 | 151.2 |

Fractions outside of the window and peaks in the window but not specified above are not collected and directed to waste.

In another aspect, the present invention provides methods for treating a subject with hyperglycemia. Such subjects include patients with Type II diabetes. The method comprises administering a composition comprising a therapeutically effective amount of TE to the subject. The present method is especially useful for treating diabetic patients who are overweight.

Dosage

TE is administered to the subject in a therapeutically effective amount. As used herein, the term “therapeutically effective amount” means the total amount that is sufficient to show a meaningful benefit, i.e., a reduction in the subject's blood glucose levels. The dosages of TE needed to obtain a meaningful result, can be determined in view of this disclosure by one of ordinary skill in the art by running routine trials with appropriate controls. Comparison of the appropriate treatment groups to the controls will indicate whether a particular dosage is effective at reducing the subject's blood glucose levels.

The amount of the TE required will depend upon the nature and severity of the condition being treated, and on the nature of prior treatments which the subject has undergone. Ultimately, the dosage will be determined using clinical trials. Initially, the clinician will administer doses that have been derived from animal studies. An effective amount can be achieved by one administration of the composition. Alternatively, an effective amount is achieved by multiple administration of the composition to the subject. In vitro, the biologically effective amount, i.e., the amount sufficient to induce glucose uptake, is administered in two-fold increments, to determine the full range of activity. The efficacy of oral, subcutaneous and intravenous administration is determined in clinical studies. Although a single administration of TE may be beneficial, it is expected that multiple doses will be preferred.

Delivery

Administration of TE, preferably is by oral administration. Although less preferred, TE may also be administered by injection.

Formulations of the present invention suitable for oral administration may be presented as discrete units such as capsules, cachets, tablets, boluses or lozenges, each containing a predetermined amount of the active compound; as a powder or granules; or in liquid form, e.g., as an aqueous solution, suspension, syrup, elixir, emulsion, dispersion, or the like. TE may be administered in the form of pills (powder or concentrated liquid in capsules), or powder form (dried powder but pressed into grains that can be consumed after putting into water (similar to drinking tea or coffee).

Formulations suitable for parenteral administration conveniently comprise a sterile preparation of the active compound in, for example, water for injection, saline, a polyethylene glycol solution and the like, which is preferably isotonic with the blood of the recipient. Useful formulations also comprise concentrated solutions or solids containing TE which upon dilution with an appropriate solvent give a solution suitable for parenteral administration.

In addition to the aforementioned ingredients, the formulations of this invention may further include one or more optional accessory ingredient(s) utilized in the art of pharmaceutical formulations, i.e., diluents, buffers, flavoring agents, colorants, binders, surface active agents, thickeners, lubricants, suspending agents, preservatives (including antioxidants) and the like.

The amount of TE required to be effective for any indicated condition will, of course, vary with the individual mammal being treated and is ultimately at the discretion of the medical or veterinary practitioner. The factors to be considered include the condition being treated, the route of administration, the nature of the formulation, the mammal's body weight, surface area, age and general condition, and the particular extract to be administered. The total daily dose may be given as a single dose, multiple doses, e.g., two to six times per day, or by intravenous infusion for a selected duration. Dosages above or below the range cited above are within the scope of the present invention and may be administered to the individual patient if desired and necessary.

The composition comprises a biologically effective amount of TE, and, optionally, a relatively inert carrier. Many such carriers are routinely used and can be identified by reference to pharmaceutical texts. The acceptable carrier is a physiologically acceptable diluent or adjuvant. The term physiologically acceptable means a non-toxic material that does not interfere with the effectiveness of the analog. The characteristics of the carrier will depend on the route of administration and particular compound or combination of compounds in the composition. Preparation of such formulations is within the level of skill in the art. The composition may further contain other agents that either enhance the activity of the analog or complement its activity. The composition may further comprise fillers, salts, buffers, stabilizers, solubilizers, and other materials well known in the art.

### EXAMPLES

The following examples are for purposes of illustration only and are not intended to limit the scope of the claims which are appended hereto. All references cited herein are specifically incorporated in their entirety herein.

### EXAMPLE 1

### Effect of a Hot Water Extract of Banaba on Glucose Uptake and Adiopogenesis

A. Materials and Methods

Materials. 3T3-L1 fibroblasts were purchased from American Type Culture Collection (ATCC, Rockville, Md.). Banaba leaves were obtained as a gift from Huagen Pharmaceuticals (Hong Kong). Dulbecco's modified Eagle's medium (DMEM) and Dulbecco's PBS (DPBS) were from Gibco Life Technologies (Grand Island, N.Y.). Fetal bovine serum (FBS) was from Atlanta Biologicals, Inc (Norcross, Ga.). Bovine insulin (IS), 3-isobuty-1-methyl-xanthine (IBMX), and dexamethasone (DEX) were from Sigma Chemical (St. Louis, Mo.). 2-deoxy-D-[³H] glucose was from Amersham Pharmacia Biotech (Piscataway, N.J.). Dianion HP-20 resin was from Mitsubishi Chemical Corporation (Tokyo, Japan). Anti-mouse GLUT4 monoclonal antibody was from Biogenesis (Brentwood, N.H.).

Banaba extract preparation. Banaba extract was prepared by the method described previously (Kakuda, T., et al. (1996) Biosci. Biotechnol. Biochem. 60: 204-208) with modifications. Banaba hot water extract (BE) was isolated by boiling 50 g of banaba tea in 1 L distilled water for 30 min, concentrate to reduce total volume by heat evaporation, followed by ultracentrifgation at 30,000 g for 30 min, filtration with a 0.45 μm filter, and freeze-drying. The BE was further separated by passing through a Dianion HP-20 resin column. The BE was loaded on the column packed with Dianion HP-20 resin, then washed with distilled water (BWE), and the absorbed fraction was eluted with methanol (BME). These two eluted fractions from the column were individually concentrated and freeze-dried. The powder of the banaba extract (BE and BME) were dissolved in sterile dH₂O, and then further sterilized with 0.2 μm filters for the adipocyte differentiation study. Unless otherwise stated, BE was used in the study.

Cell culture and adipocyte differentiation. 3T3-L1 cells were maintained in DMEM and supplemented with 10% FBS at 37° C. in a 10% CO₂ cell incubator. Preadipocyte 3T3-L1 cells were grown in 12-well plates until 2 days post confluence. The differentiation was induced as previously described (20) by addition of 1 mg/L IS, 0.5 mmol/L IBMX, and 0.25 μmol/L DEX (IS-IBMX-DEX). Two days after induction, the IS-IBMX-DEX-containing medium was replaced with medium containing 1 mg/L IS alone. The medium was subsequently replaced again with fresh culture medium (DMEM supplemented with 10% FBS) after 2 d first and then every other day thereafter. To determine the roles of banaba extract in adipocyte differentiation, BE was added to the medium to either substitute insulin (BE-IBMX-DEX), or supplement IS-IBMX-DEX (BE-IS-IBMX-DEX). The differently induced cells were assayed for glucose uptake activity 9-12 days after the initiation of induction.

Glucose uptake activity assay. Glucose uptake activity was analyzed by measuring the uptake of 2-deoxy-D-[³H] glucose as described previously (Sakoda, H, et al. (1999) Diabetes 48: 1365-1371; Garcia de Herreros, A. & Birnbaum, M. J. (1989) J. Biol. Chem. 264: 19994-19999). Briefly, confluent 3T3-L1 adipocytes grown in 12-well plates were washed twice with serum-free DMEM and incubated with 1 mL of the same medium at 37° C. for 2 h. The cells were washed 3 times with Krebs-Ringer-Hepes (KRP) buffer and incubated with 0.9 mL KRP buffer at 37° C. for 30 min. Insulin and/or BE were then added and adipocytes were incubated at 37° C. for 15 min. Glucose uptake was initiated by the addition of 0.1 ml, KRP buffer and 3.7×10⁷ Bq/L 2-deoxy-D-[³H] glucose and 1 mmol/L glucose as final concentrations. After 10 min, glucose uptake was terminated by washing the cells 3 times with cold PBS. The cells were lysed with 0.7 mL of 1% Triton X-100 at 37° C. for 20 min. The radioactivity retained by the cell lysates was determined by a scintillation counter. Nonspecific glucose uptake was measured at a glucose concentration of 100 mmol/L. Assay data were statistically analyzed using the paired Student's t-test, with P<0.05 set as the significant difference level.

Adipocyte differentiation assay. Undifferentiated 3T3-L1 preadipocytes were induced to differentiate into adipocytes as described above. The degree of the differentiation of the cells induced by different agents was evaluated by microscopic observation of lipid accumulation, as well as by the glucose uptake activities they exhibited at the end of the induction as described above. The glucose uptake assay was chosen and performed here for determination of the degree of adipocyte differentiation based on the observation that differentiated adipocytes can be induced by insulin to take up glucose whereas preadipocytes cannot.

Northern blot and Western blot analyses. Total RNA or total protein was isolated with standard procedures from 3T3-L1 cells induced by different combinations of insulin, IBMX and DEX, and BE. For detection of PPARγ2 mRNA expression, a ³²P-labeled fragment of 308 basepairs, corresponding to the nucleotides from +29 to +336 of the coding region of the PPARγ2 cDNA, was used as a probe and, 10˜tg of total RNA (isolated 144 h post induction) were used per sample. For Western blot analysis, an anti-mouse GLUT4 monoclonal antibody was used and 100 Etg of total protein (isolated 10 d post induction) were loaded per lane.

B. Results and Discussion

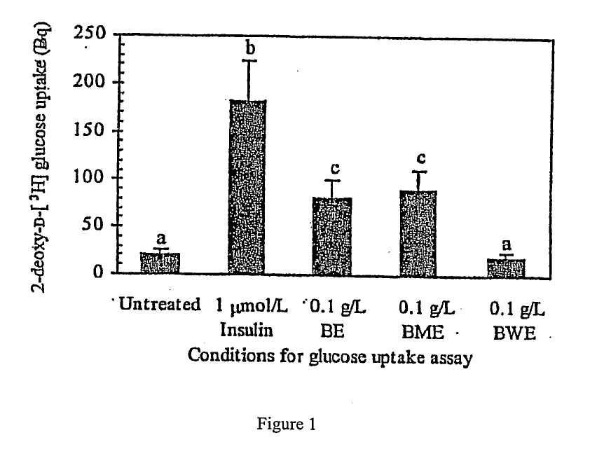

Like insulin, both the hot water extract of banaba and the banaba HP-20 methanol eluant (BE and BME, respectively) could stimulate glucose uptake in 3T3-L1 adipocytes whereas the banaba HP-20 water eluant (BWE) could not (P<0.05. FIG. 1), suggesting that the effective component(s) with glucose transport-inducing activity in the banaba extract are water-soluble but relatively non-polar. Also, the maintenance of the activity through boiling and heat evaporation during extract preparation indicates that the effective component(s) is heat stable, and is unlikely to be a protein(s).

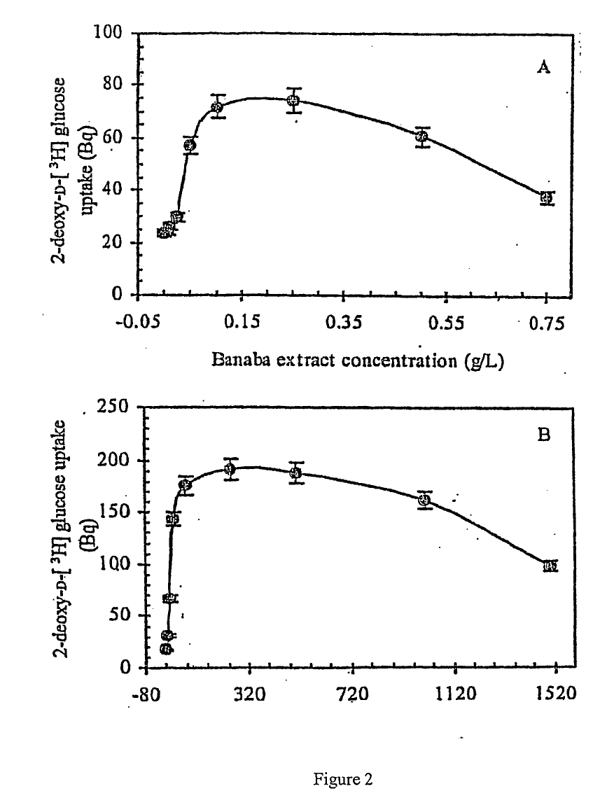

The effect of the BE's concentration on glucose uptake was then compared to that of insulin. The concentration-dependent curve of glucose uptake activity of BE (FIG. 2a) is very similar to that of insulin (FIG. 2b). The concentration range of BE that stimulates the highest glucose uptake is approximately 0.1 g/L to 0.25 g/L (FIG. 2a). The similarity between the two dose-response curves and the observation that the induction time required by BE for stimulating glucose uptake activity was similar to that of insulin, no more than 15 min (refer to the glucose uptake activity assay in Materials and Methods), suggests that BE may stimulate glucose uptake by a mechanism that is similar to that of insulin (Flores-Riveros, J. R., et al. (1993) Proc. Natl. Acad. Sci. USA 90: 512-516; Fong, J. C., et al; (1999). Cell. Signal. 11: 53-58; Baldwin, S. A., et al. (1995) Biosci. Reports 15: 419-426), and is different from the ones utilized by other known chemicals (Flores-Riveros, et al. (1993) Proc. Natl. Acad. Sci. USA 90: 512-516; Baldwin, S. A., et al (1995) Biosci. Reports 15: 419-426; Szalkowski, D., et al. (1995) Endocrinol. 136: 1474-1481.; Muralcami, N., Inoue, et al. (1997) Life Sci. 60: 1821-1831).

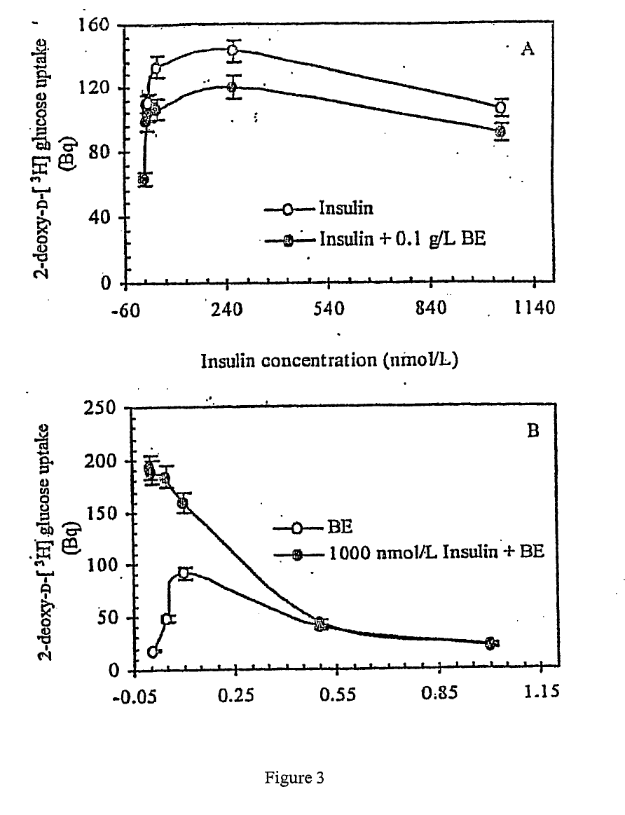

To test whether BE could further enhance insulin's glucose uptake activity, 0.1 g/L BE was added to insulin at various concentrations (0 to 1000 nmol/L). No increase in the glucose uptake was observed compared to that of insulin alone (FIG. 3a), indicating that there is no additive or synergistic effects between BE and insulin. To the contrary, reduction (P<0.05) of the glucose uptake activity by addition of BE to insulin was observed (FIG. 3b). Based upon the observations of glucose uptake-inducing activity of BE and inhibitory activity of BE on insulin-induced glucose uptake, a mechanism of action of BE may be hypothesized. BE may interact with a protein factor that is directly involved in the insulin-mediated glucose transport signaling pathway that starts with the insulin receptor and terminates with GLUT4, consequently activating the glucose uptake. On the other hand, the interaction between BE and the protein factor may structurally alter the conformation of the factor, preventing it from properly receiving the glucose uptake signal initiated from insulin-insulin receptor binding. BE apparently stimulates glucose uptake through a mechanism that is very different from the one used by other common anti-diabetic drugs such as thiazolidinediones (TZDs) (Stevenson, R. W., et al. (1996). Diabetes 45: 60-66, 30). TZDs stimulate glucose uptake using a slow and indirect mechanism by activating PPARγ, which in turn up-regulates GLUT4 gene expression (Stevenson, R. W., et al. (1996). Diabetes 45: 60-66, Park, K. S., et al. (1998) J. Clinical Endocrinol. Metab. 83: 1636-1643.; Kerstein, S., et al. (2000) Nature 405: 421-424). In contrast, BE's glucose uptake-stimulatory activity seems to be much faster, and more direct. The effective compound(s) in BE may represent a new group of chemicals that may be used as alternatives to TZDs to induce glucose uptake both in cells and in animals.

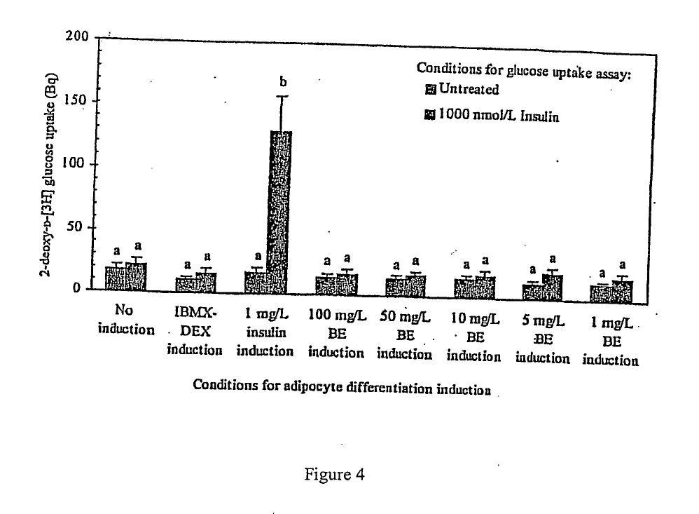

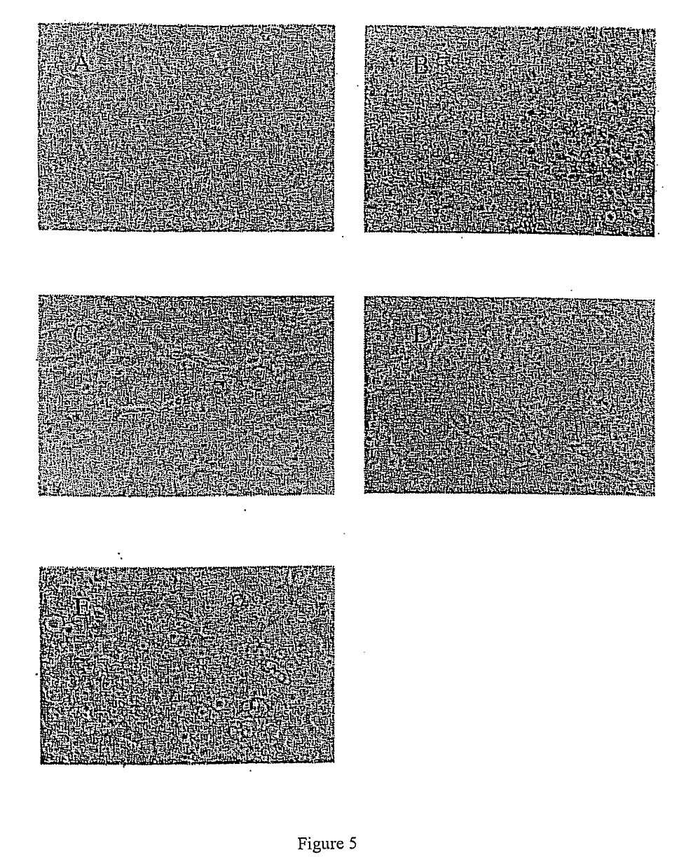

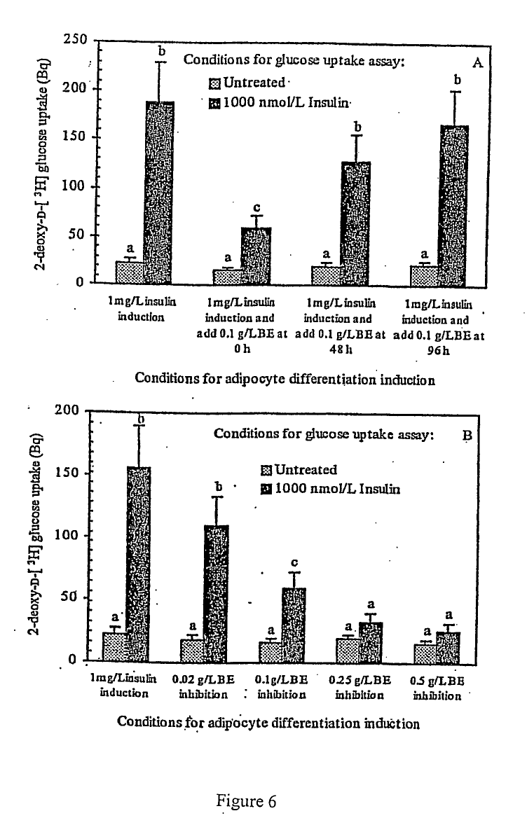

Undifferentiated 3T3-L1 preadipocytes can be converted to adipocytes by addition of a cocktail containing insulin, IBMX, and DEX (20). However, when 1 mg/L to 100 mg/L of BE was substituted for insulin and added to preadipocytes in the presence of IBMX and DEX, no adipocyte differentiation of 3T3-L1 cells was observed as revealed by glucose uptake assays (FIG. 4). This result indicates that BE does not induce adipocyte differentiation in 3T3-L1 cells. Interestingly, both BE and BME, when co-incubated with IS-IBMX-DEX, could inhibit adipogenesis whereas BWE could not (FIG. 5). This indicates that both the adipocyte differentiation inhibition activity and the glucose uptake activity originated from BME (FIG. 1). It was further shown that the inhibition of adipocyte differentiation by BE is time-dependent (FIG. 6a) and concentration-dependent (FIG. 6b). These results are consistent with the microscopic observations of fat accumulation (FIG. 5). The 3T3-L1 preadipocytes, whose differentiation was blocked by coincubation of BE and IS-IBMX-DEX, were found to retain the capacity to re-enter the differentiation process when the IS-IBMX-DEX induction cocktail was re-introduced to the cells (data not shown). It is interesting to note that BE showed an insulin-like glucose uptake-inducing activity in adipocytes but did not show an insulin-like differentiation-inducing activity in preadipocytes. This difference may be explained by the fact these two activities are from two distinct signaling pathways and that insulin receptor is involved in glucose transport process in adipocytes whereas insulin-like growth factor 1 receptor, which is homologous to insulin receptor, is used by insulin in preadipocytes for induction of differentiation (Cowherd, R. M., et al. (1999) Cell Dev. Biol. 10: 3-10; Tang, Q.-Q., & Lane, M. D. (1999). Genes Dev. 13: 2231-2241; Lane, M. D., et al. (1999) Biochem. Biophy. Res. Comm. 266: 677-683).

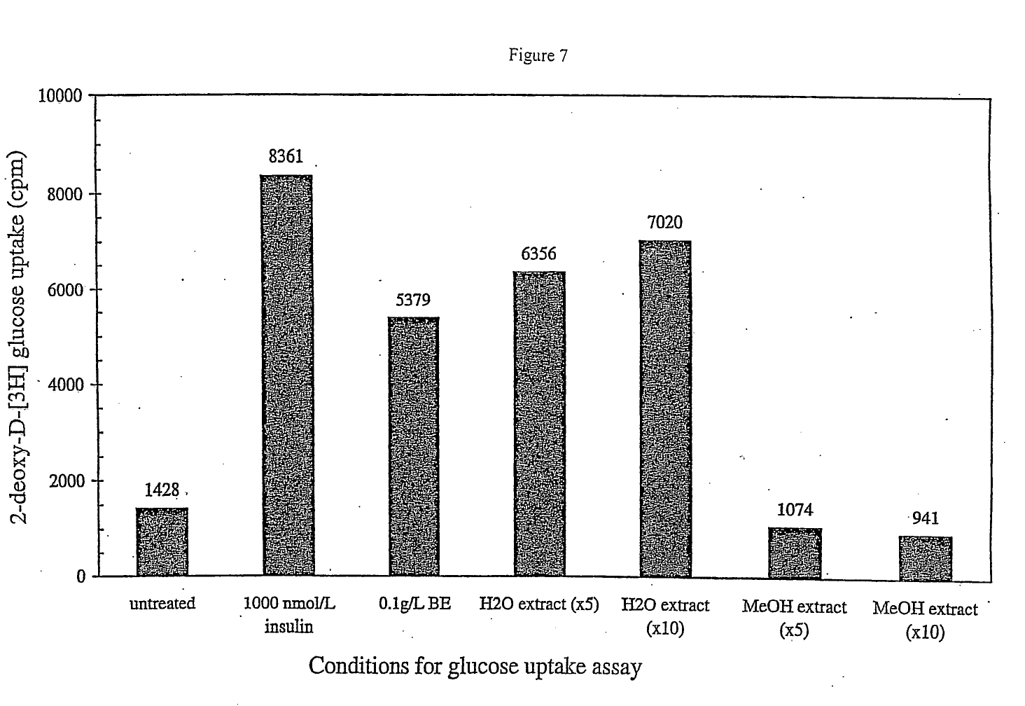

To investigate how BE inhibits adipocyte differentiation induced by IN-IBMX-DEX, two important differentiation markers, PPARγ2 and GLUT4, were used to monitor the progress of differentiation in the preadipocytes that were induced by either IBMX-DEX or IS-IBMX-DEX in the presence or absence of BE. Northern blot analysis reveals that BE greatly inhibited the mRNA expression of PPARγ2 induced either by IBMX-DEX or by IS-IBMX-DEX in a dose-dependent manner (FIG. 7A). Furthermore, as shown by a Western blot analysis, protein production of GLUT4 (˜65 kDa) was drastically inhibited in the cells induced by IS-IBMX-DEX in the presence of BE than the cells induced in the absence of BE (FIG. 7B). These results are consistent with our other differentiation inhibition results studied with glucose uptake assays (FIGS. 4 & 6), and are not inconsistent with other glucose uptake assays (FIGS. 1-3) since BE's glucose uptake-inducing activity was examined in fully differentiated adipocytes (FIGS. 1-3) whereas BE's GLUT4 inhibitory effect was observed in undifferentiated cells (FIG. 7B). GLUT4 expression was used as an indicator of differentiation in this study to monitor the ability of BE to induce differentiation in preadipocytes, not glucose transport in adipocytes. All of these results suggest that the specific target of the differentiation inhibition exerted by BE is PPARγ2 or factor(s) that directly or indirectly regulates the expression of PPARγ2. Further experiments are necessary to determine the action site of the inhibition. The identity and function of the 43 kDa protein (FIG. 7B) are unclear at this time. The presence of such a protein band in the Western blot was likely due to the specific monoclonal antibody, and possibly the cells used in this study.

Antidiabetic drugs such as insulin or TZDs up-regulate both glucose transportation and lipid biosynthesis in adipocytes (Stevenson, R. W., et al. (1996 Diabetes 45: 60-66.; Park, K. S., et al. (1998). J. Clinical Endocrinol. Metab. 83: 1636-1643). Weight gain is a frequent side effect of insulin therapy in type II diabetic patients (Laville, M. & Andreelli, F. (2000). Diabetes Metab. 26 (Suppl 3): 42-5). Therefore, drugs with glucose-lowering activity, but lacking adipogenic activity are highly desirable. The effective component(s) of BE seems to have such an advantageous combination

### EXAMPLE 2

### Characterization of Methanol Extracts and Water Extracts of Banaba

Banaba leaves were extracted twice with 500 mL of hexane, twice with 500 mL of methanol (MeOH extract), and then twice with 500 mL of H₂O(H₂O extract) at 37° C. Adipocytes in 24-well plates were incubated for 15 min with the MeOH extracts or H₂O extracts, or with insulin and banaba hot water extract (BE) as positive control, or without treatment as negative control. Following treatment, the cells were assayed for 2-deoxy-D-[³1H]glucose uptake. As shown in FIG. 7, the banaba H₂O extract stimulated glucose uptake in 3T3-L1 adipocytes, but the MeOH extract did not.

Undifferentiated 3T3-L1 preadipocytes were incubated with either insulin, banaba hot water extract (BE), or different concentrations of banaba H₂O extract in the presence of dexamethasone (DEX) and 3-isobutyl-1-methylxanthine (IBMX). Following ten days of treatment, the degree of adipocyte differentiation was assayed by the glucose uptake activities of the cells. As shown in FIG. 8, the banaba H₂O extract inhibited adipocyte differentiation of 3T3-L1 cells induced by insulin and IBMX and DEX.

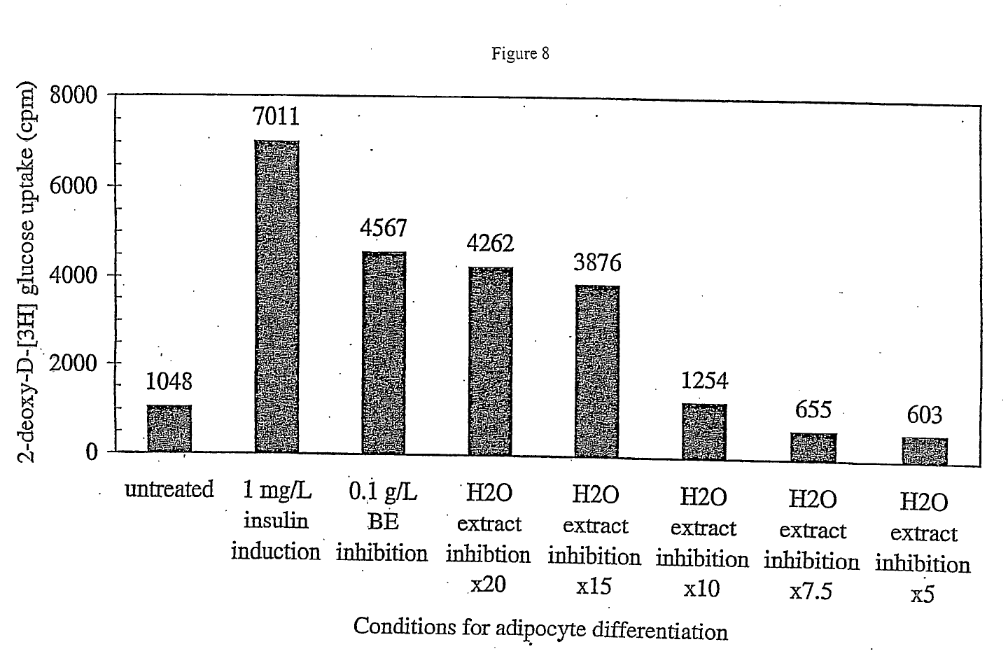

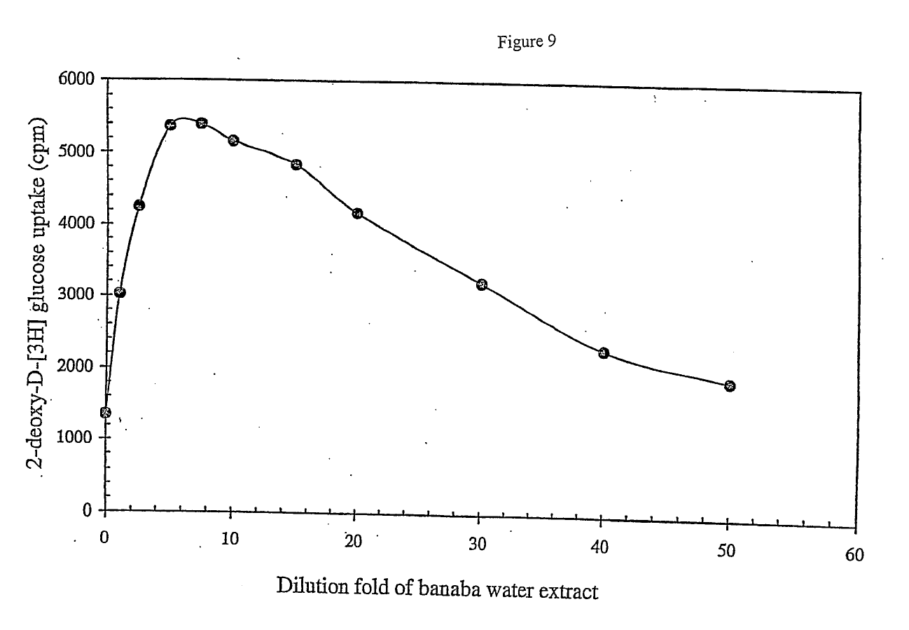

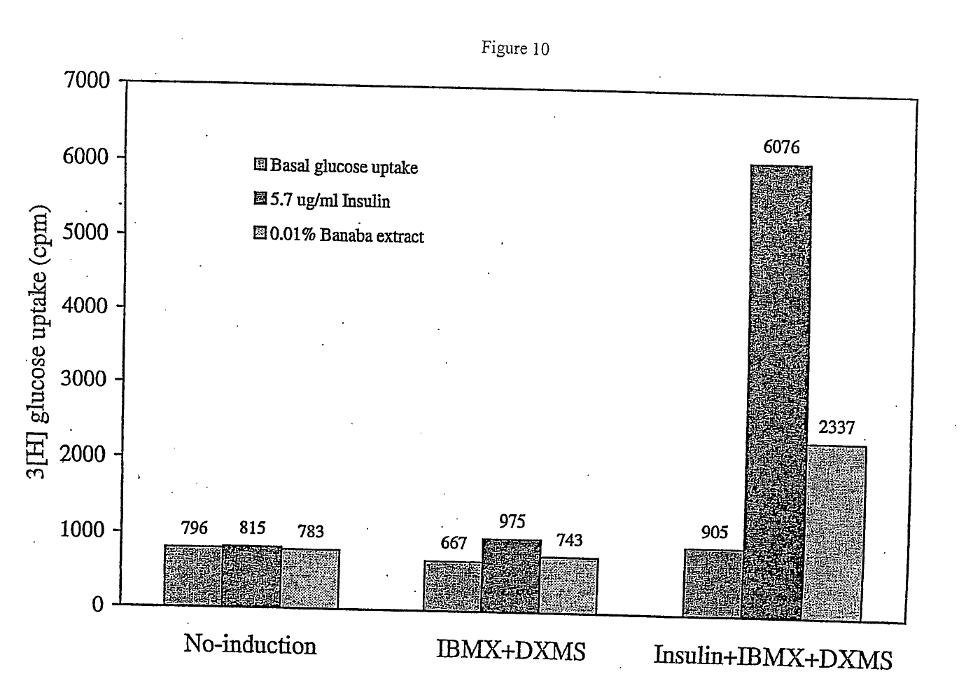

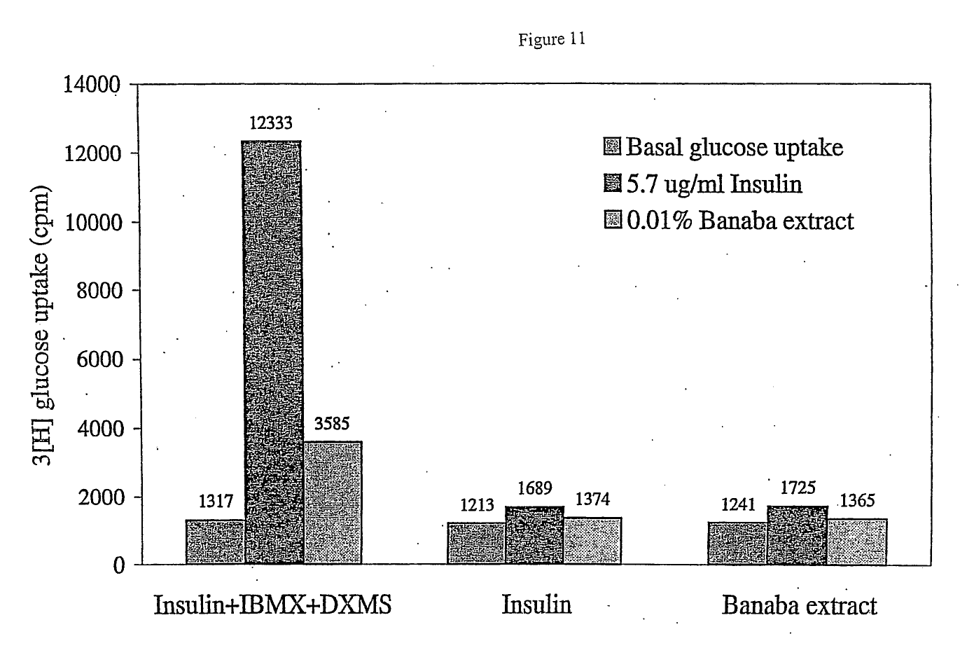

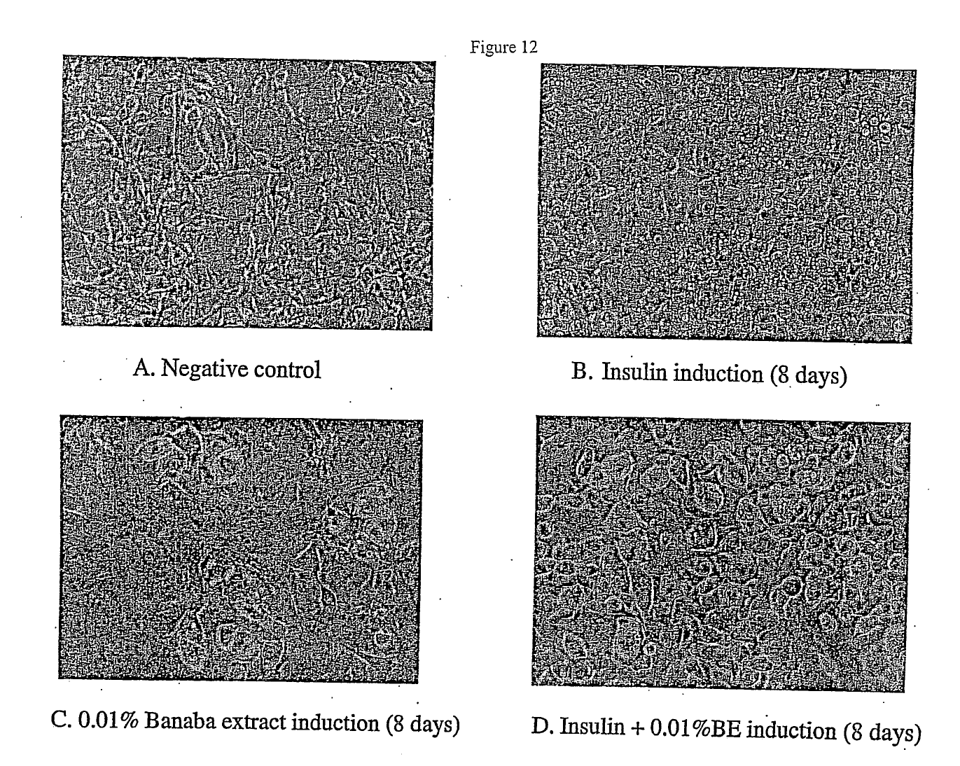

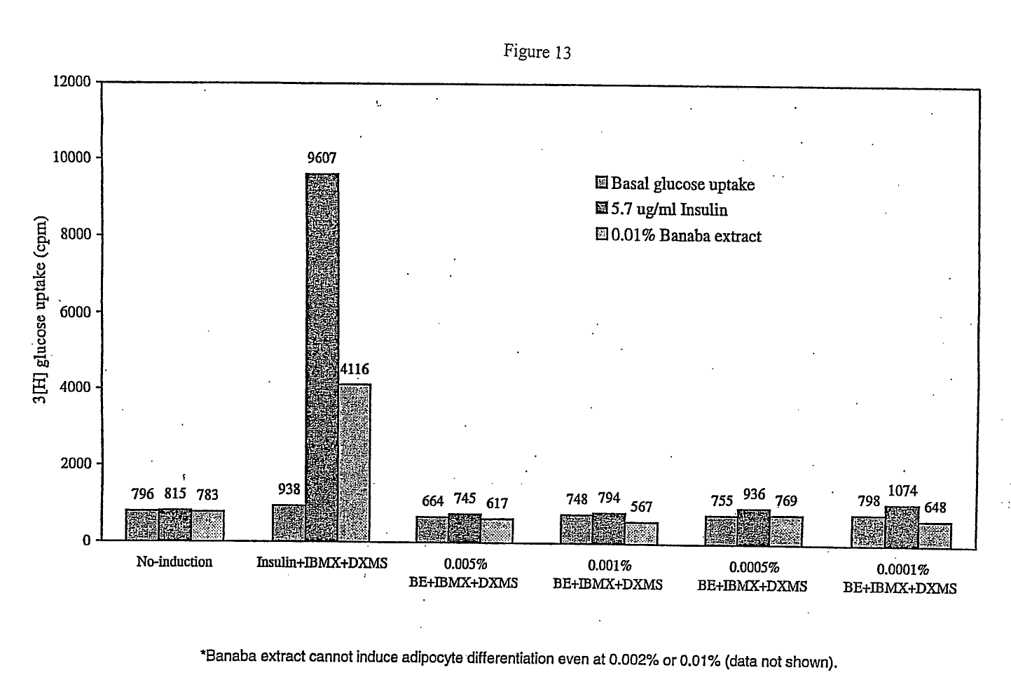

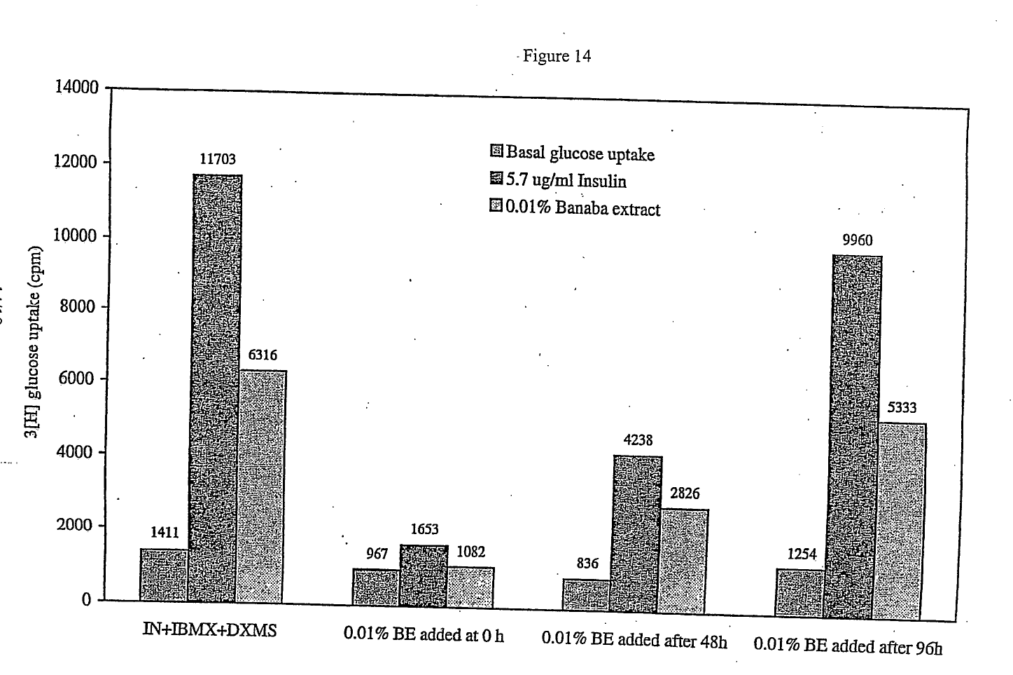

Differentiated 3T3-L1 cells in 24-well plates were incubated with different concentrations of the banaba H₂O extract for 15 minutes, and then assayed for 2-deoxy-D[³H]glucose uptake. As shown in FIG. 9, the concentration-dependent curve of glucose uptake activity of banaba H₂O extract is very similar to those of insulin and banaba hot water extract (BE).

These results show that the gulucose-uptake stimulatory activity (FIG. 7) and the adipocyte differentiation inhibition activity (FIG. 8) are both in the banaba water extract and not the methanol extract of banaba. The triterpenoid corosolic acid is hydrophobic and is expected to be in the methanol extract of banaba, suggesting that corosolic acid is not one of the active factors in the banaba water etract.

Thin layer chromatography (TLC) identification of the compounds in the methanol extract and the H₂O extract of banaba was carried out on silica gel in solvent of 5-10% MeOH—CHCl₃ with detection by spraying a saturated solution of ceric sulfate in 65% sulfuric acid or FeCl₃ ethanol solution. Pure ursolic acid was used as the triterpenoid control and commercial tannic acid mix was used as a tannic acid control. The results indicated that triterpeonids were in the methanol extract of banaba and not in the banaba water extract. The results also indicated that the water extract of banaba contained tannic acids.

The methanol and water extracts of banaba were also subjected to Q-TOF electrospray mass spectra analysis. The results confirmed that the methanol extract of banaba contained corosolic acid. However, methanol extract of banaba tea does not show glucose uptake activity, indicating that corosolic acid is not in the effective fraction.

The banaba tea hot water extract (BE) was also reacted with BSA and gelatin, reagents known to precipitate tannic acids. No glucose uptake stimulatory activity was observed in the remaining extract after BSA and gelatin precipitation of BE. Collectively, these results suggest that one of more of the active components in BE are tannic acids.

### EXAMPLE 3

### Treatment of Normal Mice with BE

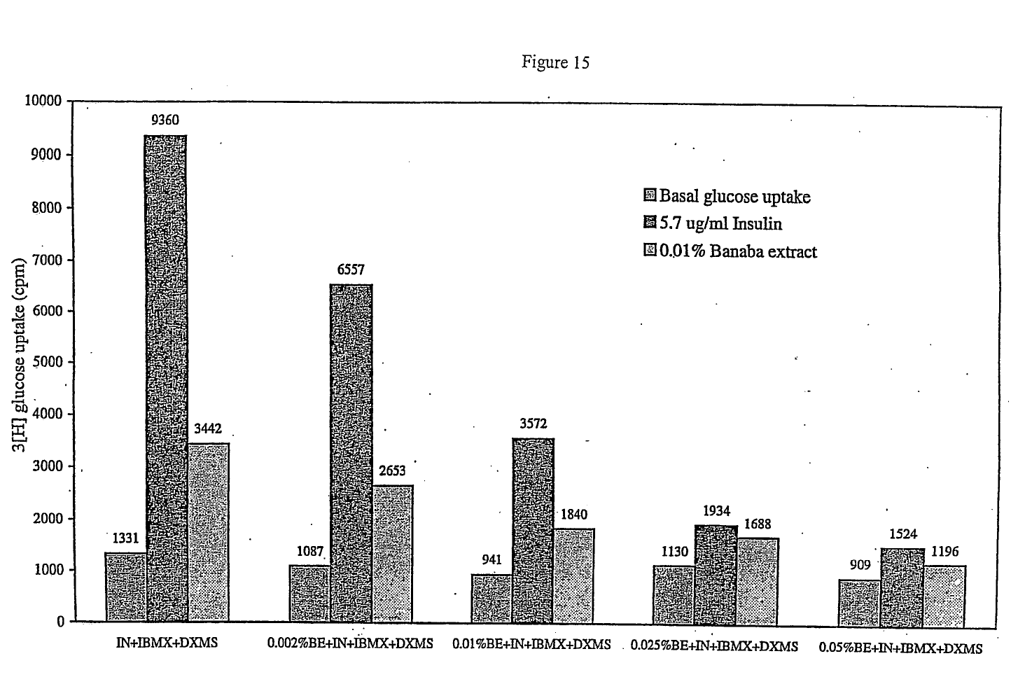

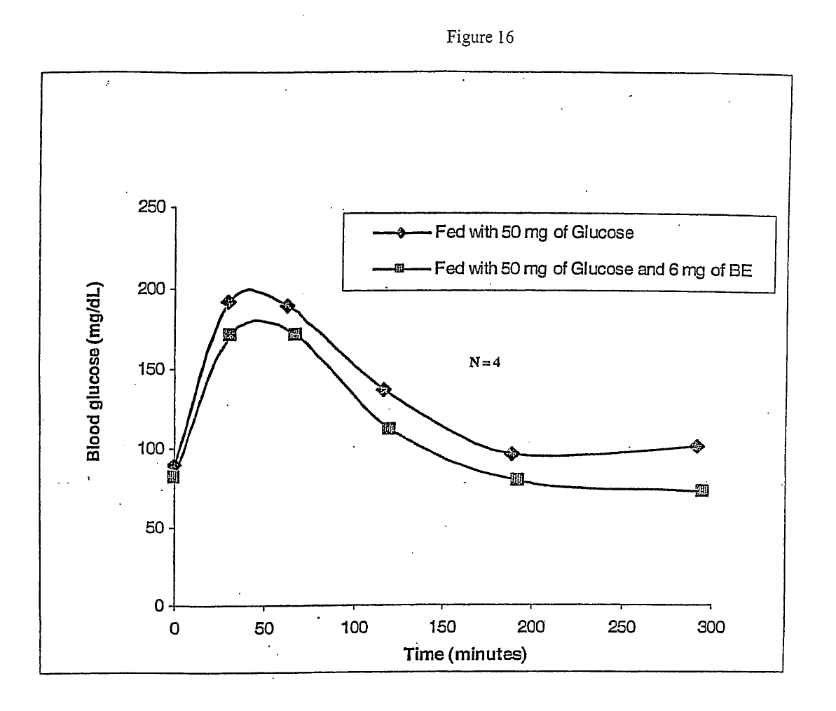

Normal mice of three month of age, with an average weight of 24 grams, were used to assess the effect of BE on glucose uptake in vivo. Mice were fed with regular food and water prior to treatment with BE. At time 0, the mice were fed with a glucose solution or a solution of glucose and banaba extract (BE). Total volume of glucose or glucose plus BE solutions fed to the mice was 100 μL. At various intervals after treatment, blood was collected through the tail vein. Six μL of total blood was used for the blood glucose testing. One Touch Basic complete diabetes monitoring system, from Johnson & Johnson company, was used for measuring blood glucose levels. Experimental and control groups were randomly chosen, and 4 mice were used per group. The experimental data were plotted with the Microsoft Excel program. As shown in FIG. 15, feeding normal mice with BE concurrently with 50 mg glucose lowers the peak blood glucose levels that occur when the mice are fed glucose alone, i.e., without BE.

### EXAMPLE 4

### Preparation of Partially-Purified Extracts of Banaba

A hot water extract of banaba tea leaves was prepared as described above in Example 1. The extract was further purified into active fractions using HPLC methods as described below. The conditions of the first HPLC separation were as follows:

HPLC system is Beckman System Gold consisting of a 125 solvent module, a 168 PDA detector and a 508 autosampler. A Beckman Ultrasphere C-18 reversed phase column (4.6 mm×250 mm I.D., 5 m) was used. The detection wavelengths were set at 210 nm, 260 nm and 370 nm. Eluent A was a mixture of water and 0.1% trifluoroacetic acid, eluent B was a mixture of acetonitrile and 0.1% trifluoroacetic acid. Separation was achieved with following linear gradients from A to B in 60 minutes at a flow rate of 1 mL/min:

Isocratic A for 5 min, 100:0 to 90:10 in 10 min, isocratic for 10 min, 90:10 to 80:20 in 10 min, isocratic for 10 min, 80:20 to 50:50 in 5 min, isocratic for 10 min.

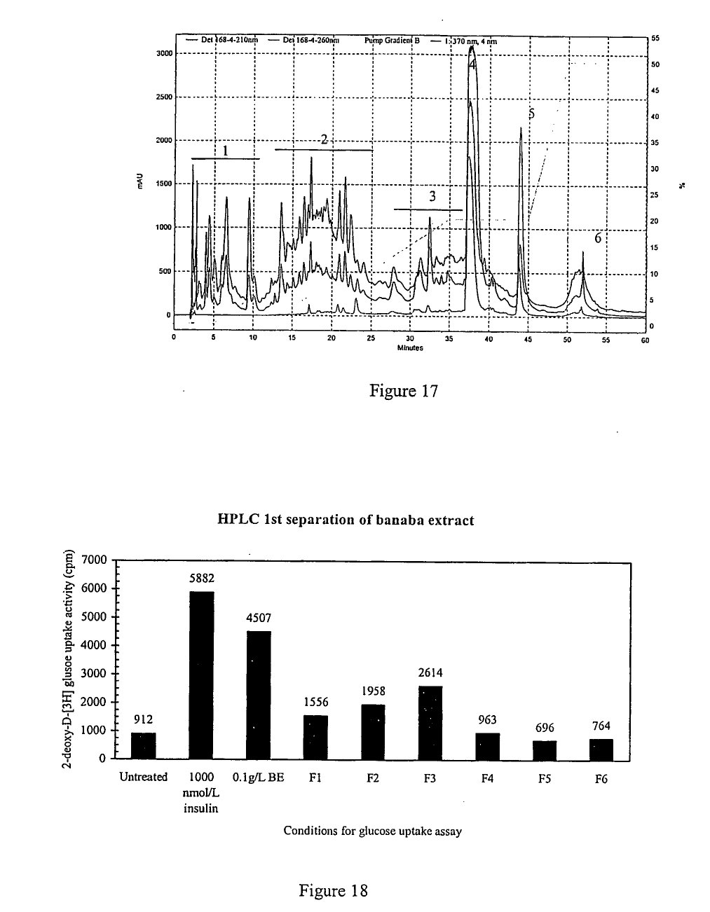

A profile of the fractions generated by HPLC running method 1 are shown in FIG. 17. HPLC fractions 1-6 were isolated from the extract and tested for glucose uptake stimulatory activity. Adipocytes in 12-well plates were incubated with 0.1 g/L of Fractions 1-6, or with insulin and banaba hot water extract (BE) as positive control, or without treatment as negative control. The cells were then assayed for 2-deoxy-D-[³H]glucose uptake. As shown in FIG. 18, HPLC fractions 2 and 3 (F2 and F3) contained significant biological activity.

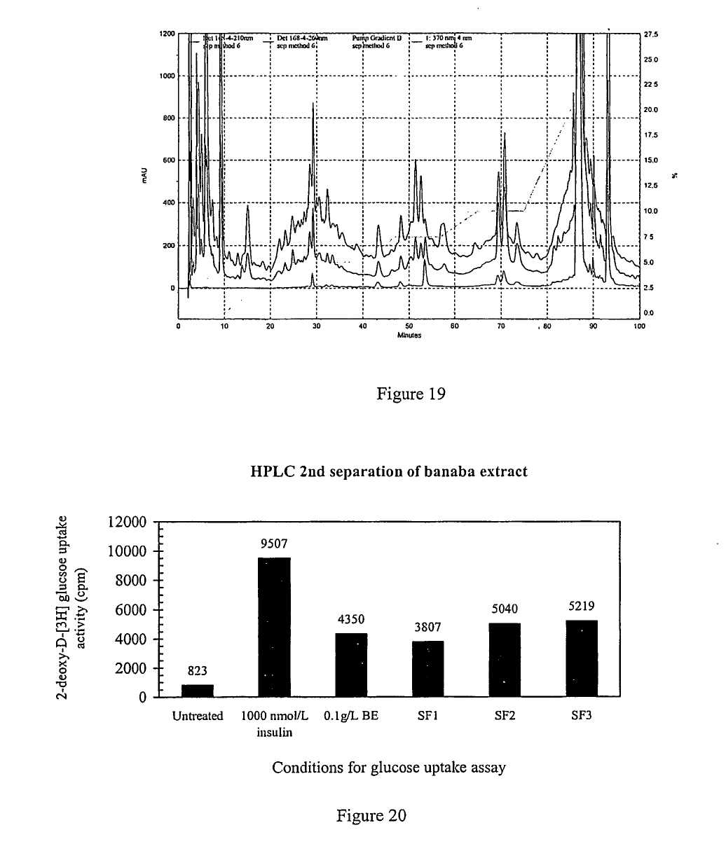

Running conditions of HPLC for the banaba hot water extract were changed from HPLC running method 1 to HPLC running method 2 as described below so that fractions F2 and F3 from running HPLC running method 1 could be further separated into subfractions as shown in FIG. 19. Fraction F2, as shown in FIG. 17, was further separated into subfractions (SF) SF1 and SF2, while fraction F3 was further separated into sub-fraction SF3.

HPLC system is Beckman System Gold consisting of a 125 solvent module, a 168 PDA detector and a 508 autosampler. A Beckman Ultrasphere C-18 reversed phase column (4.6 mm×250 mm I.D., 5 μm) was used. The detection wavelengths were set at 210 nm, 260 nm and 370 nm. Eluent A was a mixture of water and 0.1% trifluoroacetic acid, eluent B was a mixture of acetonitrile and 0.1% trifluoroacetic acid. Separation was achieved with following linear gradients from A to B in 115 minutes at a flow rate of 1 mL/min

Isocratic A for 10 min, 100:0 to 95:5 in 15 min, isocratic for 15 min, 95:5 to 92.5:7.5 in 7 min, isocratic for 10 min, 92.5:7.5 to 90:10 in 8 min, isocratic for 10 min, 90:10 to 80:20 in 10 min, isocratic for 20 min, 80:20 to 50:50 in 5 min and then isocratic for 5 min. Subfractions SF1, SF2, and SF3 as generated by HPLC running method 2 were isolated and assayed for their glucose uptake stimulatory activity. Adipocytes in 12-well plates were incubated for 15 min with 1 g/L of HPLC fractions SF1, SF2, SF3, or with insulin and banaba hot water extract (BE) as positive control, or without treatment as negative control, then assayed for 2-deoxy-D-[³H]glucose uptake. As shown in FIG. 20, all three subfractions generated by HPLC running method 2 are active with sub-fractions SF2 and SF3 being the most active.

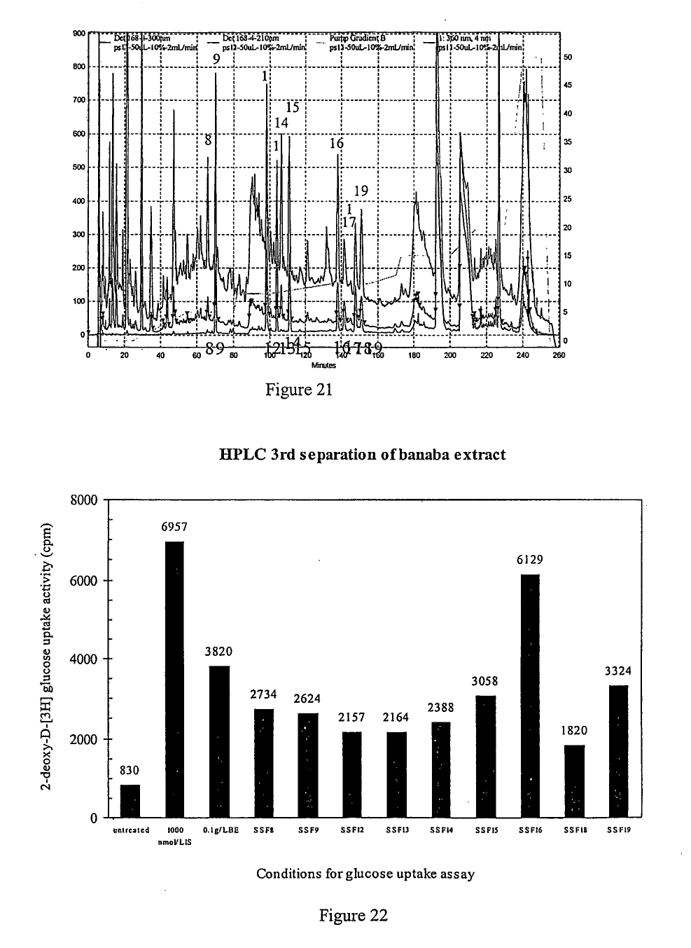

HPLC running conditions for the hot water extract were changed to HPLC running method 3 described below so that subfractions SF1, SF2, and SF3 could be further separated into sub-subfractions as shown in FIG. 21.

HPLC system is Beckman System Gold consisting of a 125 solvent module, a 168 PDA detector and a 508 autosampler. A Beckman Ultrasphere C-18 reversed phase column (10.0 mm×250 mm I.D., 5 μm) was used. The detection wavelengths were set at 210 nm, 300 nm and 370 nm. Eluent A was a mixture of water and 0.1% trifluoroacetic acid, eluent B was a mixture of acetonitrile and 0.1% trifluoroacetic acid. A Foxy Jr. fraction collector from ISCO was used to collect individual peaks in timed windows. Separation was achieved with following linear gradients from A to B in 250 minutes at a flow rate of 2 mL/min, Isocratic A for 25 min, 100:0 to 95:5 in 30 min, isocratic for 25 min, 95:5 to 91.8:8.2 in 2 min, isocratic for 18 min, 91.8:8.2 to 90:10 in 35 min, isocratic for 20 min, 90:10 to 88.5:11.5 in 15 min, 88.5:11.5 to 85:15 in 3 min, isocratic for 27 min, 85:15 to 80:20 in 15 min, isocratic for 15 min, 80:20 to 50:50 in 10 min and then isocratic for 10 min.

Among the 26 sub-subfractions shown in FIG. 20, 9 were collected to sufficient amounts for biological assays. Adipocytes in 12-well plates were incubated for 15 min with 1 g/L of the isolated HPLC sub-subfractions, or with insulin and banaba hot water extract (BE) as positive control, or without treatment as negative control, then assayed for 2-deoxy-D-[³H]glucose uptake. As shown in the FIG. 21, all 9 sub-subfractions (i.e., subfractions 8, 9 12, 13, 14, 15, 16, 18, and 19) are active, with sub-subfractions SSF15, SSF16, and SSF19 being more active than the other sub-subfractions and sub-subfraction SSF16 being significantly more active than any others. Thus, sub-subfraction SSF16 generated by HPLC running method 3 is the most potent compound for stimulating glucose transport activity in 3T3-L1 cells.

Q-TOF electrospray mass spectra analysis was performed on sub-subfractions generated by HPLC running method 3. The results showed the most active sub-subfractions had molecular weights of 939 grams/mole and 935 grams/mole. These values are comparable to the molecular weight of penta-O-galloyl-D glucose, which has a calculated molecular weight of 940 grams/mole and the ellagitannin casuarictin, which has a calculated molecular weight of 936 grams/mole.

1. A partially-purified plant extract for treating subjects with hyperglycemia;
wherein said extract is a partially-purified extract of a plant that comprises hydrolysable gallotannins;
wherein said extract comprises a plurality of water soluble compounds having a molecular weight of from 800 to 1500 grams/mole; and
wherein said extract is substantially free of triterpenoids.

2. The partially purified plant extract of claim 1 wherein said extract comprises 1,2,3,4,6-penta-O-galloyl-D-glucose.

3. The partially purified plant extract of claim 2 wherein said extract is a partially purified extract of banaba.

4. The partially purified plant extract of claim 3 wherein said extract is a partially-purified water extract of banaba.

5. The partially purified plant extract of claim 3 wherein said extract comprises a compound having a molecular weight of 936 grams/mole.

6. A method for preparing a partially- purified banaba extract for treating hyperglycemia in a subject, comprising:
preparing a water extract of banaba;
fractionating said water extract into a plurality of fractions;
isolating one or more active fractions from said plurality of fractions to provide said partially-purified banaba extract;
wherein said one or more active fractions comprise a plurality of compounds having a molecular weight of from 800 to 1500 grams/mole and;
wherein said one or more active fractions are free of triterpenoids; and
wherein said one or more active fractions stimulate glucose uptake by adipocytes.

7. The method of claim 6 wherein said water extract is a water extract of banaba leaves.

8. The method of claim 6 wherein said partially purified banaba extract comprises 1,2,3,4,6-penta-O-galloyl-D-glucose.

9. The method of claim 6 wherein said water extract of banaba is fractionated into a plurality of fractions by HPLC.

10. A partially-purified extract for treating subjects with hyperglycemia, wherein said extract is prepared by the method of claim 6.

11. A method for treating or preventing diabetes in a mammalian subject, comprising:
administering a therapeutically effective amount of the partially-purified plant extract of claim 1 to a subject in need of the same.,

12. The method of claim 11 wherein said partially-purified plant extract is a partially purified water extract of banaba.

13. The method of claim 11 wherein said partially purified plant extract comprises 1,2,3,4,6-penta-O-galloyl-D-glucose.

14. The method of claim 11 wherein said partially purified plant extract is administered to the subject orally or by injection.

15. A method of reducing elevated blood glucose levels in a subject in need thereof comprising:
administering the partially-purified plant extract of claim 1 to the subject.

16. The method of claim 15 wherein said partially purified plant extract is a partially-purified water extract of banaba.

17. The method of claim 15 wherein said partially purified plant extract comprises 1,2,3,4,6-penta-O-galloyl-D-glucose.

18. The method of claim 15 wherein said partially-purified plant extract is administered to the subject orally or by injection.

19. A method of treating a subject exhibiting one or more of obesity, type II diabetes, glucose intolerance, or hyperglycemia, comprising
administering the partially-purified plant extract of claim 1 to the subject.

20. The method of claim 19 wherein said partially-purified plant extract is an extract of banaba.

21. The method of claim 19 wherein said partially purified plant extract comprises 1,2,3,4,6-penta-O-galloyl-D-glucose.

22. The method of claim 19 wherein said partially purified plant extract is administered to the subject orally or by injection.

23. The method of claim 19 wherein said partially-purified extract inhibits differentiation of preadipocytes to adipocytes.

24. A partially-purified plant extract for treating subjects with hyperglycemia;
wherein said extract is a partially-purified extract of a plant that comprises hydrolysable gallotannins;
wherein said extract comprises a plurality of water soluble compounds having a molecular weight of from 800 to 1500 grams/mole; and
wherein said extract comprises less than 0.5% by weight of triterpenoids.

25. The partially-purified plant extract of claim 24, wherein said extract comprises less than 0.1% by weight of triterpenoids.
